(19) United States
(12) Patent Application Publication
(10) Pub. No.: US 2025/0064521 A1
(43) Pub. Date: Feb. 27, 2025

(54) DISTANCE AND SIZE MEASUREMENT SYSTEM FOR POP-DUSTING EFFICIENCY

(71) Applicant: Lumenis Ltd., Yokneam Illit (IL)

(72) Inventors: Asaf Granot, Lotem (IL); Vitaly Rondel, Hadera (IL)

(73) Assignee: Lumenis Ltd., Yokneam Illit (IL)

(21) Appl. No.: 18/813,706

(22) Filed: Aug. 23, 2024

Related U.S. Application Data
(60) Provisional application No. 63/578,820, filed on Aug. 25, 2023.

(51) Int. Cl. A61B 18/26; A61B 17/00; A61B 18/00; A61B 18/22; A61B 90/00; G01S 7/481; G01S 17/08

(52) U.S. Cl. CPC A61B 18/26; G01S 7/4814; G01S 7/4818; G01S 17/08; A61B 2017/00199; A61B 2018/00511; A61B 2018/00982; A61B 2018/2222; A61B 2090/061

(57) ABSTRACT

Described herein are laser emitting medical devices and techniques implementable by laser emitting medical devices to determine and display a pop-dusting efficiency of a lasing treatment where a distal end of an optical fiber is disposed in a liquid environment. The devices and techniques provide to determine the pop-dusting efficiency based on the average distance of a target from the distal end of the optical fiber, as well as the “strikes per second” as calculated by a computing system.

### CROSS-REFERENCE TO RELATED APPLICATIONS

This application claims the benefit of U.S. Provisional Patent Application Ser. No. 63/578,820 filed on Aug. 25, 2023, the disclosure of which is incorporated herein by reference.

### TECHNICAL FIELD

The present disclosure generally relates to a surgical laser system. Particularly, but not exclusively, the present disclosure relates to surgical laser systems used in lithotripsy procedures.

### BACKGROUND

Medical lasers are used in a variety of procedures. Among several of the procedures, laser energy is directed towards a target using a fiber as a conduit for the laser energy. One such procedure, to address renal calculi (e.g., kidney stones) is ureteral endoscopy, or lithotripsy. An endoscopic probe, with a camera or other sensor, is inserted into the patient's urinary tract to locate the calculi for removal. In endoscopic lithotripsy, the probe also includes an optical fiber, which conducts a laser beam to disintegrate the calculi as they are found.

An ideal lithotripsy procedure is fast, precise, and thorough. The medical practitioner minimizes the time that the endoscope is inserted, directs all the discharged laser energy into target calculi, and assures that no large stones or fragments remain. However, even with the advancements in technology, modern medical devices do not have a method to determine when all calculi (or stones) are small enough without relying on a human scanning the treatment area with the endoscope camera. Furthermore, a pop-dusting procedure produces stone dust, which reduces and/or entirely obscures visibility in the treatment environment. Given the reduced visibility, the physician may not lase efficiently. As such, the present disclosure provides for determining and displaying to the physician an efficiency measurement of the pop-dusting procedure as well as an indication that the procedure may be concluded.

### BRIEF SUMMARY

This summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The present disclosure provides an endoscopic lithotripsy device for determining both the efficiency of a pop-dusting lithotripsy procedure and for determining when the sizes of the stone fragments are small enough to conclude the procedure. In general, the disclosure provides a lithotripsy device configured to measure the distance between a distal tip of a fiber and a stone fragment. The lithotripsy device configured to determine a pop-dusting efficiency measure.

In some embodiments, the disclosure can be implemented as a computer implemented method. The method can comprise receiving, at a processor, a first electrical signal generated by a first light sensor, the first electrical signal comprising an indication of a power of a light received at the first light sensor, wherein the light received at the first light sensor corresponds to laser light generated by a laser source and emitted from a distal end of an optical fiber towards one or more targets; receiving, at the processor, a second electrical signal generated by a second light sensor, the second electrical signal comprising an indication of a power of a light received at the second light sensor, wherein the light received at the second light sensor corresponds to a laser light reflected from at least one or more targets; determining, at the processor during a time period, a plurality of distances based on the second electrical signal and the first electrical signal, each of the distances corresponding to a distance between the distal end of the optical fiber and at least one of the one or more targets; and determining, at the processor, a pop-dusting efficiency based on the plurality of distances.

In further embodiments of the computer implemented method, the optical fiber is coupled to a laser console comprising the processor.

In further embodiments of the computer implemented method, the laser console comprises a lasing system comprising: the light sensor; a laser source arranged to generate the laser light; and a beam splitter arranged to direct a portion of the laser light from the laser source to the optical fiber and arranged to direct the laser light reflected from the one or more targets to the light sensor.

In further embodiments of the computer implemented method, the laser console comprises the second light sensor and wherein the beam splitter is further arranged to direct a portion of the laser light from the laser source to the second light sensor.

In further embodiments of the computer implemented method, the laser source comprises either a Holmium-based lasing medium or a Thulium-based lasing medium.

In further embodiments of the computer implemented method, the laser console further comprises an optical head comprising at least a lens arranged to couple the laser light to the optical fiber.

In further embodiments of the computer implemented method, the lasing system further comprises a second laser source and the method further comprises: receiving, at a processor, a third electrical signal generated by the first light sensor, the third electrical signal comprising an indication of a power of a light received at the first light sensor that corresponds to laser light generated by the second laser source and emitted from the distal end of the optical fiber towards the one or more targets; receiving, at the processor, a fourth electrical signal generated by the second light sensor, the fourth electrical signal comprising an indication of a power of a light received at the second light sensor that corresponds to laser light reflected from at least one of the one or more targets; and determining, at the processor during the time period, the plurality of distances based on the first electrical signal, the second electrical signal, the third electrical signal and the fourth electrical signal.

In further embodiments, the computer implemented method comprises identifying, by the processor, a reference in a lookup table corresponding to the distance, wherein the lookup table is stored in a memory coupled to the processor, and wherein the lookup table correlates a distance of the target with the first electrical signals and second electrical signals; and determining the distance of the target from the distal end of the optical fiber based on the reference.

In further embodiments, the computer implemented method comprises executing, by the processor, a machine learning (ML) model to generate an inference of the distance of a target from the distal end of the optical fiber, wherein the machine learning model is executed with at least the first electrical signal and second electrical signals as input.

In further embodiments of the computer implemented method, the optical fiber is arranged to be inserted through a working channel of a ureteroscope.

In further embodiments, the computer implemented method comprises determining the distance of the target from the distal end of the optical fiber; identifying in a lookup table a reference corresponding to a predetermined distance threshold; comparing the distance of the target to the predetermined distance threshold; counting distances less than the threshold as strikes; and updating the pop-dusting efficiency based on strikes per second.

In further embodiments of the computer implemented method, as the plurality of distances decreases, the pop-dusting efficiency increases.

In further embodiments of the computer implemented method, as the plurality of distances increases, the pop-dusting efficiency decreases.

In further embodiments of the computer implemented method, the laser console comprises user interface elements which may facilitate display, execution, interaction, manipulation, or operation of program components through textual or graphical facilities.

In further embodiments of the computer implemented method, the user interface elements include a pop-dusting efficiency gauge, which is configured to display the pop-dusting efficiency.

With some embodiments, the disclosure can be implemented as a system for a laser console. The system can comprise a processor; and a memory device coupled to the processor, the memory device having instructions stored thereon, which instructions when executed by the processor, cause the system to receive a first electrical signal generated by a first light sensor, the first electrical signal comprising an indication of a power of a light received at the first light sensor, wherein the light received at the first light sensor corresponds to laser light generated by a laser source and emitted from a distal end of an optical fiber towards one or more targets; receive a second electrical signal generated by a second light sensor, the second electrical signal comprising an indication of a power of a light received at the second light sensor, wherein the light received at the second light sensor corresponds to a laser light reflected from at least one or more targets; determine, during a time period, a plurality of distances based on the second electrical signal and the first electrical signal, each of the distances corresponding to a distance between the distal end of the optical fiber and at least one of the one or more targets; and determine a pop-dusting efficiency based on the plurality of distances.

In further embodiments of the system, the optical fiber is coupled to the laser console.

In further embodiments of the system, the instructions, when executed by the processor, further cause the system to identify a reference in a lookup table corresponding to the distance, wherein the lookup table is stored in a memory coupled to the processor, and wherein the lookup table correlates a distance of the target with the first electrical signals and second electrical signals; and determine the distance of the target from the distal end of the optical fiber based on the reference.

In further embodiments of the system, the laser console comprises a lasing system comprising the light sensor; a laser source arranged to generate the laser light; and a beam splitter arranged to direct a portion of the laser light from the laser source to the optical fiber and arranged to direct the laser light reflected from the one or more targets to the light sensor.

In further embodiments of the system, the laser console comprises the second light sensor and wherein the beam splitter is further arranged to direct a portion of the laser light from the laser source to the second light sensor.

In further embodiments of the system, the laser source comprising either a Holmium-based lasing medium or a Thulium-based lasing medium.

In further embodiments of the system, the laser console further comprises an optical head comprising at least a lens arranged to couple the laser light to the optical fiber.

In further embodiments of the system, the lasing system further comprises a second laser source and wherein the instructions, when executed by the processor, further cause the system to receive a third electrical signal generated by the first light sensor, the third electrical signal comprising an indication of a power of a light received at the first light sensor that corresponds to laser light generated by the second laser source and emitted from the distal end of the optical fiber towards the one or more targets; receive a fourth electrical signal generated by the second light sensor, the fourth electrical signal comprising an indication of a power of a light received at the second light sensor that corresponds to laser light reflected from at least one of the one or more targets; and determine, at the processor during the time period, the plurality of distances based on the first electrical signal, the second electrical signal, the third electrical signal and the fourth electrical signal.

In further embodiments of the system, the instructions, when executed by the processor, further cause the system to execute a machine learning (ML) model to generate an inference of the distance of a target from the distal end of the optical fiber, wherein the machine learning model is executed with at least the first electrical signal and second electrical signals as input.

In further embodiments of the system, the optical fiber is arranged to be inserted through a working channel of a ureteroscope.

In some embodiments, the disclosure can be implemented as a computer-readable memory storage device comprising a plurality of instructions, which when executed by a processor of a medical laser console, cause the medical laser console to receive a first electrical signal generated by a first light sensor, the first electrical signal comprising an indication of a power of a light received at the first light sensor, wherein the light received at the first light sensor corresponds to laser light generated by a laser source and emitted from a distal end of an optical fiber towards one or more targets; receive a second electrical signal generated by a second light sensor, the second electrical signal comprising an indication of a power of a light received at the second light sensor, wherein the light received at the second light sensor corresponds to a laser light reflected from at least one or more targets; determine, during a time period, a plurality of distances based on the second electrical signal and the first electrical signal, each of the distances corresponding to a distance between the distal end of the optical fiber and at least one of the one or more targets; and determine a pop-dusting efficiency based on the plurality of distances.

In further embodiments of the computer-readable memory storage device, the instructions, when executed by the processor further cause the medical laser console to determine the distance of the target from the distal end of the optical fiber; identify in a lookup table a reference corresponding to a predetermined distance threshold; compare the distance of the target to the predetermined distance threshold; count distances less than the threshold as strikes; and update the pop-dusting efficiency based on strikes per second.

In further embodiments of the computer-readable memory storage device, as the plurality of distances decreases, the pop-dusting efficiency increases.

In further embodiments of the computer-readable memory storage device, as the plurality of distances increases, the pop-dusting efficiency decreases.

In further embodiments of the computer-readable memory storage device, the medical laser console comprises a display and the instructions, when executed by the processor further cause the medical laser console to generate one or more graphical information elements comprising indications of the pop-dusting efficiency; and cause the display to display the graphical information elements as part of a user interface.

In further embodiments of the computer-readable memory storage device, the one or more graphical information elements comprise a pop-dusting efficiency gauge, which is configured to display the pop-dusting efficiency.

### BRIEF DESCRIPTION OF THE DRAWINGS

To easily identify the discussion of any element or act, the most significant digit or digits in a reference number refer to the figure number in which that element is first introduced.

FIG. 1A illustrates a computer system in communication with a laser system. The laser system emitting laser light through an optical fiber to target(s) and receiving reflected laser light back.

FIG. 1B illustrates an optic fiber emitting laser light onto a target and receiving reflected light back.

FIG. 2 illustrates an example lasing system.

FIG. 3A illustrates an optical fiber emitting laser light when no targets are in range.

FIG. 3B illustrates an optical fiber emitting light when a target is in range with low impact.

FIG. 3C illustrates an optical fiber emitting laser light when a target is in range with high impact.

FIG. 3D illustrates where some fragments have been broken down, and large ones remain.

FIG. 3E illustrates where all fragments have been broken down.

FIG. 4 illustrates a method to determine a pop-dusting efficiency.

FIG. 5 illustrates another a method to determine a pop-dusting efficiency.

FIG. 6 illustrates yet another a method to determine a pop-dusting efficiency.

FIG. 7A illustrates an example graphical information element indicating a pop-dusting efficiency.

FIG. 7B illustrates the graphical information element of FIG. 7A in alternative detail.

FIG. 8 illustrates computer executable instructions in accordance with one embodiment.

FIG. 9 illustrates a block diagram of a computing environment.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

As introduced above, the present disclosure provides a lithotripsy device configured to provide feedback on pop dusting efficiency and completion. When treating renal calculi with a lithotripsy procedure, the stone fragments are often “dusted” or broken up into progressively smaller and smaller fragments. This is commonly referred to as “pop-dusting” or “pop-corning” The challenge and a common question for the physician is when the fragments are “small enough” to be considered dust, which will then be removed naturally away from the kidneys via the urinary tracts. The term pop-dusting or pop-corning arises from the resemblance of the procedure of making popcorn. The method involves locking a bundle of stones in a calix corner and then firing a laser treatment beam to creates turbulence. The turbulence causes the stone fragments to be turned or tumbled into the “range” of the laser. When a fragment is in the line of sight of the fiber, the fragment will be impacted by the treatment beam. As such, fragments are continually being impacted, or “struck” by the treatment beam, which progressively breaks the stones into smaller and smaller fragments.

Currently, there is no known method to assess the efficiency of a pop-dusting procedure. For example, current lasing consoles do not provide information regarding whether the particles size is small enough for the procedure to be considered complete. It is to be appreciated that the lack of indication for the efficiency of the procedure may result in a longer than necessary procedure, and in extreme cases, can result in heating up of the kidney due to excess energy that is being delivered to the kidney with no real effect on the stones. Further, the lack of an indication for when the particles size is small enough may result in a lower stone free rate, which can result in the reappearance of kidney stones necessitating another lithotripsy procedure, sometimes within as little as a few months from the initial procedure.

Currently, physicians determine the pop-dusting efficiency based on both experience and the “blur” in the endoscope image captured during the procedure, which are both very subjective observations. The present disclosure provides objective measurements that can be measured by a lithotripsy system and provided to the physician to give the physician both an indication of the pop-dusting efficiency and whether the stone particle sizes are smaller than a threshold or desired level.

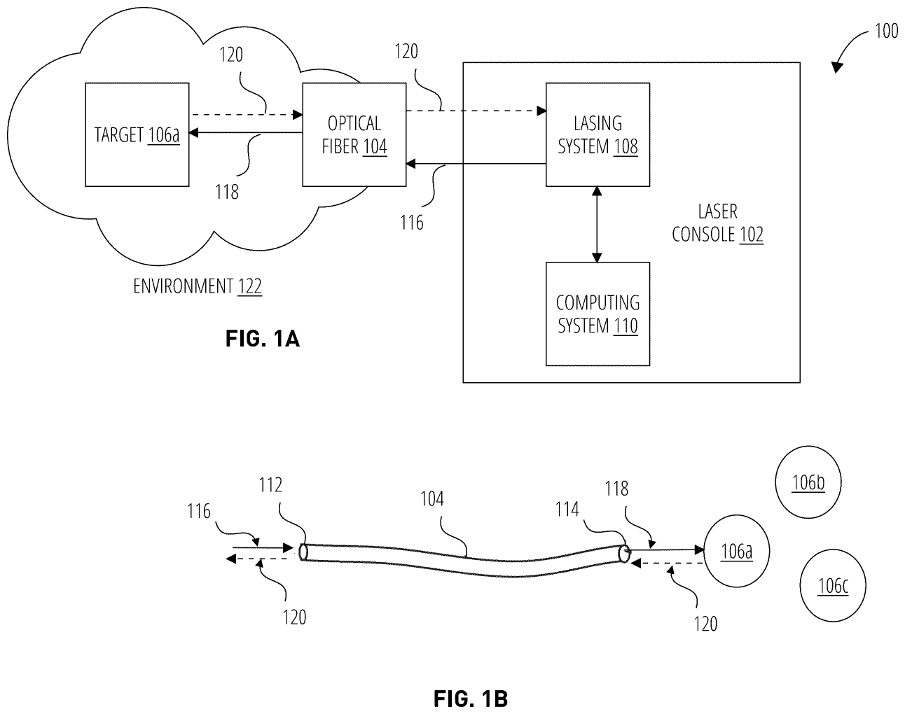

FIG. 1A and FIG. 1B show an exemplary lithotripsy procedure system 100 for determining a distance between a distal tip of a fiber and a target, which can be used (as described more fully below) to determine a pop-dusting efficiency and determining when the sizes of the stone fragments are small enough to conclude the procedure, in accordance with some embodiments of the present disclosure. In some embodiments, the exemplary lithotripsy procedure system 100 comprises a laser console 102 and an optical fiber 104 configured to emit laser light (e.g., emitted laser light 118) towards a target or targets (e.g., target 106a, 106b and 106c). The laser console 102 can include a lasing system 108 and a computing system 110.

In some embodiments, the targets 106a, 106b and 106c are stones or stone fragments to be dusted. The stone fragments will typically be located within the urinary tracts of a subject, which are to be treated via a pop-dusting procedure. In some embodiments, the subject may be a human being or an animal. During operation, the optical fiber 104 is coupled to the laser console 102 and inserted into an environment 122 of the target (e.g., via a ureteroscope, or the like) and placed proximate to the targets 106a, 106b, and 106c, where laser energy can be generated by the laser console 102 and directed towards the targets 106a, 106b and 106c via the optical fiber 104.

As depicted more fully in FIG. 1B, the optical fiber 104 comprises a proximal end 112 and a distal end 114. The proximal end 112 is the end of the optical fiber 104 coupled to the laser console 102 and through which light beams enter while the distal end 114 is the end of the optical fiber 104 through which light beams are emitted and via which light beams can be directed onto the target 106a. For example, FIG. 1B depicts laser light 116 entering the optical fiber 104 at the proximal end 112 and propagating through length of the optical fiber 104. A portion of laser light 116 exits the distal end 114 of the optical fiber 104 as emitted laser light 118 and is directed towards one of the targets. In this example, emitted laser light 118 is incident on the target 106a. Furthermore, when emitted laser light 118 is incident on a target (e.g., target 106a, or the like), a portion of the emitted laser light 118 will be reflected from the target 106a and transmitted back up the optical fiber 104 as reflected laser light 120. It is to be appreciated that other portions of the laser light 116 may be reflected up the optical fiber 104 (e.g., by the distal end 114 of the optical fiber 104 due to the interface between the distal end 114 and the environment 122, or the like). This is described in greater detail below.

Laser light 116 can be generated by lasing system 108. Lasing system 108 may include, but is not limited to, solid-state lasers, gas lasers, diode lasers, and fiber lasers. As an illustrative example, lasing system 108 can be configured to generate laser light 116 using a Holmium-based lasing medium or a Thulium-based lasing medium. Lasing system 108 can comprises optical components which may include, but are not limited to, a lasing medium, pump lights, polarizers, beam splitters, beam combiners, light detector, wavelength division multiplexers, collimators, circulators, lenses, or other such optical components arranged in various combinations to provide laser light 116.

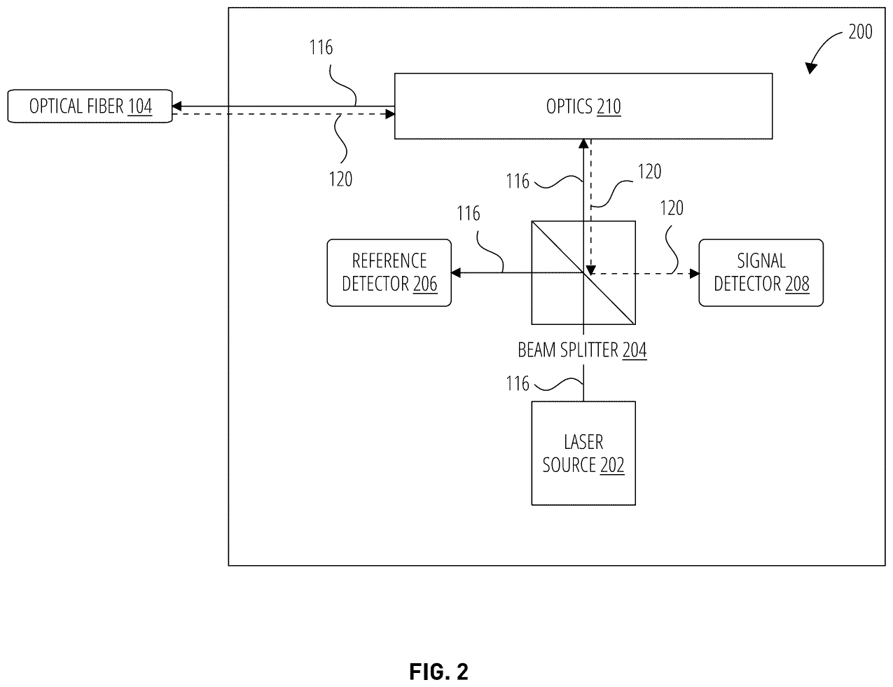

FIG. 2 illustrates an example lasing system 200, which can be implemented as lasing system 108 of lithotripsy procedure system 100 of FIG. 1A. Lasing system 200 can include a laser source 202, a beam splitter 204, a reference detector 206, a signal detector 208, and optics 210. As described above, laser source 202 can be arranged to generate laser light 116 via several different lasing mechanisms, such as, for example, using a Holmium lasing medium, using a Thulium lasing medium, or the like.

Laser light 116 can be directed to beam splitter 204, which splits the laser light 116 to direct a portion of laser light 116 to optics 210 and a portion of laser light 116 to reference detector 206. Beam splitter 204 may include any of a variety of optical components used to split incident light at a designated ratio into two separate beams.

Optics 210 can comprise any of a variety of optical component arranged to condition and direct laser light 116 from beam splitter 204 to optical fiber 104 and direct reflected laser light 120 from optical fiber 104 to beam splitter 204. Optics 210 can include polarizers, beam combiners, collimators, circulators, lenses, etc.

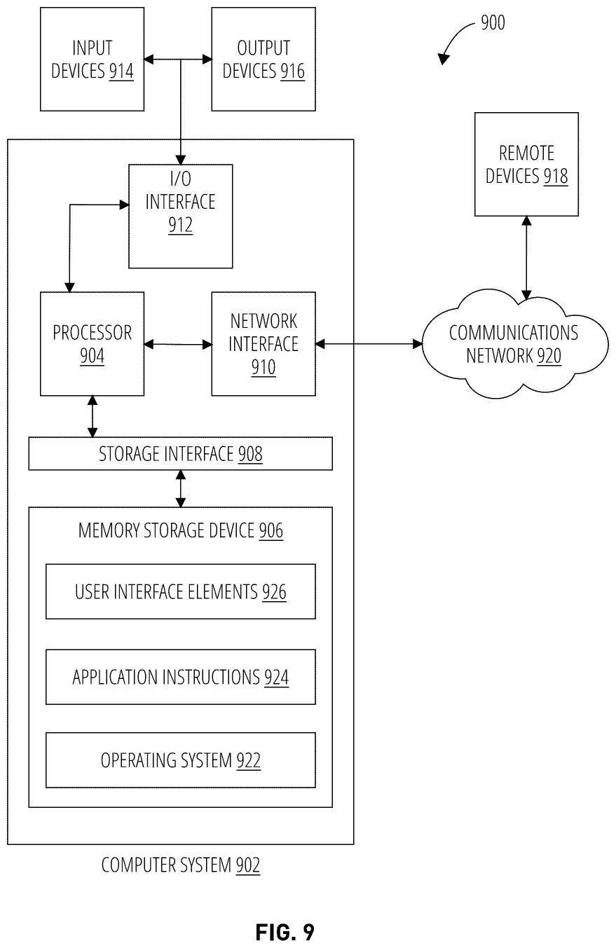

From optics 210, reflected laser light 120 is directed to beam splitter 204, which reflects reflected laser light 120 to signal detector 208. Reference detector 206 and signal detector 208 can be any of a variety of light detectors. In general, such light detectors may include devices that detect and/or measure characteristics of light beams and encode the detected and/or measured characteristics in electrical signals. For example, light detectors may detect the specific type of light beams (as preconfigured), and convert the light energy associated with the detected light beams into electrical signals. These electrical signals can be communicated to a computing device (e.g., computing system 110, or the like) to determine the power of emitted laser light 118 as described herein. In general, the computing system 110 can include circuitry arranged to determine a size and distance of targets, as well as strikes per second. This is described in greater detail below, for example, with reference to the example computing environment 900 shown in FIG. 9.

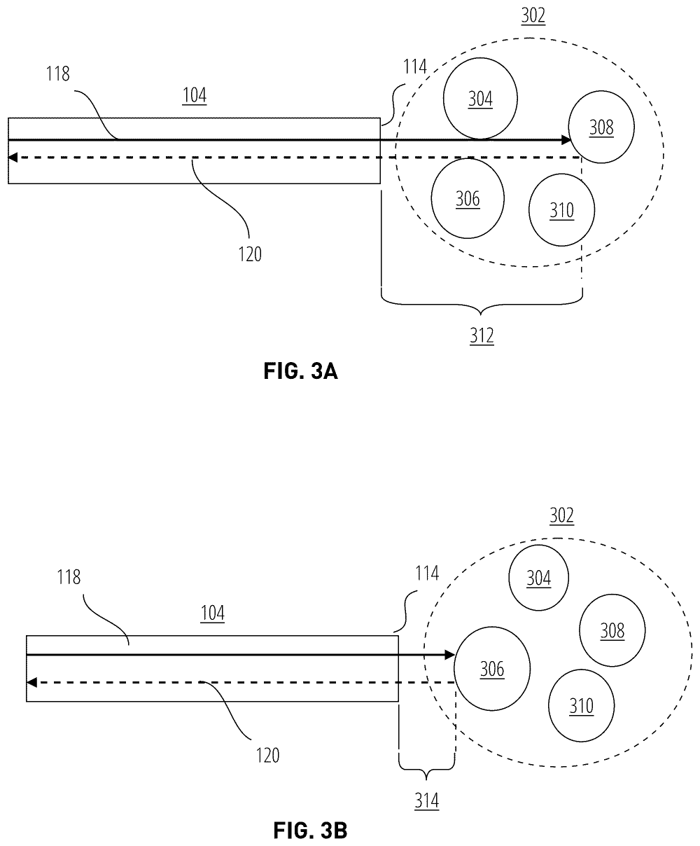

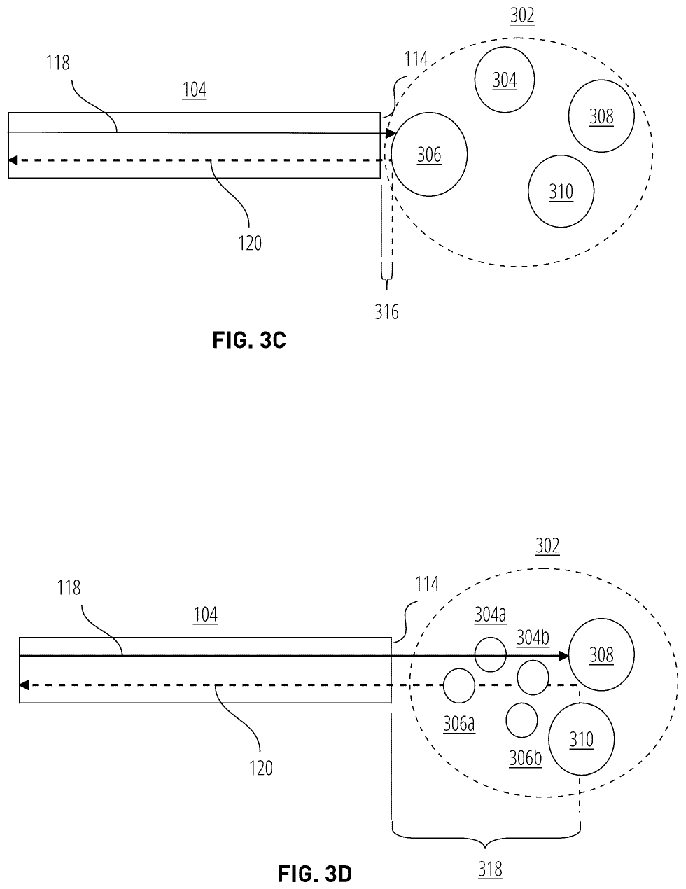

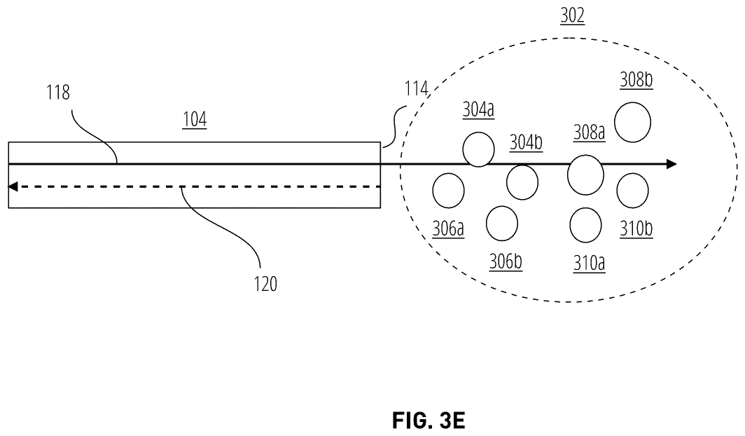

As stated above, during a pop-dusting procedure, a bundle or group of stones are locked into a corner and laser light (e.g., emitted laser light 118) is directed towards the bundle causing the stones to move about within the bundle. As the stones move about within the bundle, the particular stone with which the optical fiber 104 is directed will change and further the distance between the distal end 114 of the optical fiber 104 and the stone at which is directed will change. For example, FIG. 3A depicts the optical fiber 104 with distal end 114 disposed in the environment 122 and directed at a bundle of stone fragments 302. During operation, laser light 116 (not shown) is transmitted down the optical fiber 104, a portion of which is emitted into the environment 122 as emitted laser light 118. When the emitted laser light 118 is incident on one of the stone fragments within bundle of stone fragments 302, and a portion of the emitted laser light 118 is reflected up the fiber as reflected laser light 120. The magnitude or energy level of the reflected laser light 120 will depend upon the distance between the distal end 114 and the stone fragment. FIG. 3A illustrates bundle of stone fragments 302 comprising stone fragments 304, 306, 308 and 310. In the example shown in FIG. 3A, the emitted laser light 118 is incident on stone fragment 308, resulting in reflected laser light 120 being transmitted up optical fiber 104.

The distance 312 between distal end 114 and the stone fragment 308 (e.g., stone fragment with which the emitted laser light 118 is incident, or the like) can be determined based on the intensity of the laser light 116 and reflected laser light 120 (e.g., as described above with respect to FIG. 1A, FIG. 1B and FIG. 2).

As noted above, due to the emitted laser light 118 being directed at the bundle of stone fragments 302, the stone fragments within the bundle will tumble or move about. Accordingly, the stone within the “line-of-sight” of the optical fiber 104 will change. For example, FIG. 3B illustrates stone fragment 306 being within the line-of-sight of the optical fiber 104. As such, emitted laser light 118 is incidence on the stone fragment 306. Further, reflected laser light 120 is reflected from stone fragment 306, a portion of which is transmitted back up optical fiber 104 as described herein. The distance 314 between distal end 114 and stone fragment 306 can again be determined based on the intensity of emitted laser light 118 and reflected laser light 120.

When a target is near the distal end 114 of the optical fiber 104, the interface between distal end 114 changes from distal end 114 and environment 122.

Further, as the stone fragments within bundle of stone fragments 302 move about, the distance between a particular stone fragment (e.g., stone fragment 306, or the like) and the distal end 114 of the optical fiber 104 will change. For example, FIG. 3C again illustrates emitted laser light 118 incident upon stone fragment 306 of bundle of stone fragments 302. However, the distance 316 between distal end 114 and stone fragment 306 has changed from the example shown in FIG. 3B.

The different distances and time durations can be indicative of the efficiency of the lasing or pop-dusting. For example, the distance 312 may be too great to enable sufficient energy from emitted laser light 118 to reach the stone fragment and as such, the stone fragment may not be ablated or dusted in a significant manner. Conversely, distance 314 may be close enough for the stone fragment to be ablated or dusted, but possibly not in as significant a way as when the stone fragment is as close as distance 316.

During operation, as the stone fragments within the bundle of stone fragments 302 churn or tumble about, the number of times a stone fragment is within “range” (e.g., less than a threshold distance from distal end 114 of optical fiber 104, or the like) can be counted. This is referred to herein as a “strike.” It is to be appreciated that as the stone fragments are ablated or dusted by emitted laser light 118, the size of the stone fragments will decrease, and the number of stone fragments can increase. Additionally, if the duration of time between strikes increases, it may be indicative of the stone fragments within the bundle of stone fragments churning or tumbling around slowly. This slow movement means that the stone fragments may not be ablated or dusted in an efficient manner. Once a stone fragment shrinks below a certain, or threshold size, the stone fragment will be transparent to the lasing system 200 and a distance between the stone fragment will not be measured. As such, even where the distal end 114 is directed at the stone fragment, a strike will not be counted.

FIG. 3D illustrates an example where stone fragment 304 and stone fragment 306 have been dusted into smaller stone fragments 304a, 304b, 306a and 306b. However, these stone fragments may be small enough in size that the lasing system 200 may not measure the distance between distal end 114 of optical fiber 104 and these smaller stone fragments but will instead only measure the distance 318 between distal end 114 of optical fiber 104 and stone fragment 308, even where the stone fragments are in the line-of-sight of the optical fiber 104. Accordingly, the number of times a strike is counted in a given measure of time (e.g., 1 second, or the like) will decrease as the size of the stone fragments decreases.

The strike count per measure of time will continue to decrease until all stone fragments are broken down to a sufficiently small size. As discussed above, stone fragments below a certain size will not be recognized by the computing system 110 as they don't reflect a significant portion of emitted laser light 118. FIG. 3E illustrates an example where all stone fragments in bundle of stone fragments 302 are broken down (or dusted) to less than a threshold size. As depicted, all stone fragments have been broken down into smaller stone fragments (e.g., stone fragments 304a, 304b, 306a, 306b, 308a, 308b, 310a and 310b). As these stone fragments are all less than a threshold size, no reflected laser light 120 is depicted, despite emitted laser light 118 being directed into the bundle of stone fragments 302.

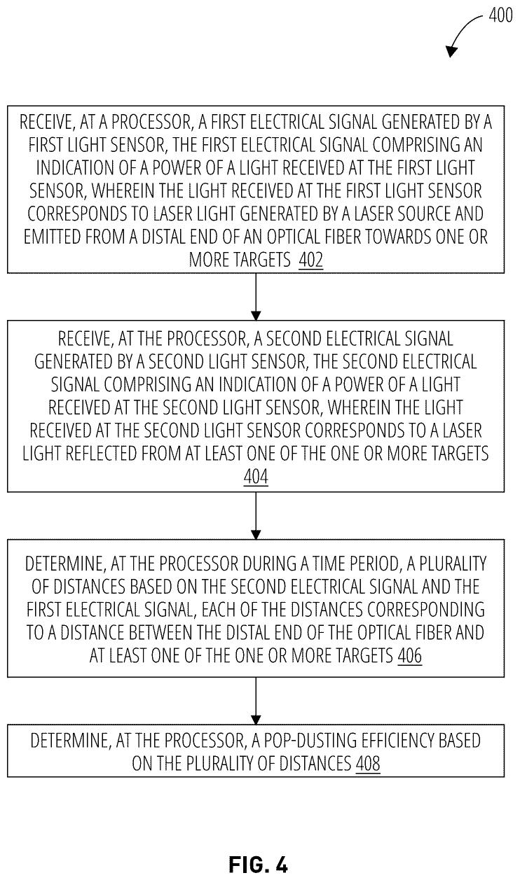

FIG. 4 illustrates a method 400 for determining a pop-dusting efficiency, according to at least one embodiment of the present disclosure. Method 400 can be implemented by a computing device of a lithotripsy system, such as, for example, computing system 110 of lithotripsy procedure system 100. Further, method 400 will be described with reference to lithotripsy procedure system 100 as well as lasing system 200 and computing environment 900 for clarity of presentation. However, it is to be appreciated that method 400 could be implemented by a system different than that described herein. Method 400 can begin at block 402. At block 402 “receive, at a processor, a first electrical signal generated by a first light sensor, the first electrical signal comprising an indication of a power of a light received at the first light sensor, wherein the light received at the first light sensor corresponds to laser light generated by a laser source and emitted from a distal end of an optical fiber towards one or more targets” a first electrical signal from a light sensor indicative of a power of a laser light emitted towards one or more targets can be received. For example, computing system 110 can receive an electrical signal from reference detector 206 comprising an indication of an intensity of laser light 116. As another example, processor 904 can execute application instructions 924 stored in memory storage device 906, which when executed can cause processor 904 to receive an electrical signal from reference detector 206 where the electrical signal is indicative of an intensity of laser light 116.

Continuing to block 404 “receive, at the processor, a second electrical signal generated by a second light sensor, the second electrical signal comprising an indication of a power of a light received at the second light sensor, wherein the light received at the second light sensor corresponds to a laser light reflected from at least one of the one or more targets” a second electrical signal from a light sensor indicative of a power of a laser light reflected from one of the one or more targets can be received. For example, computing system 110 can receive an electrical signal from signal detector 208 comprising an indication of an intensity of reflected laser light 120. As another example, processor 904 can execute application instructions 924, which when executed can cause processor 904 to receive an electrical signal from signal detector 208 where the electrical signal is indicative of an intensity of reflected laser light 120.

In some embodiments, the lasing system may include two laser sources, which can, for example, operate sequentially. The two sensors as described above, may measure the intensity of light emitted by both laser sources. For example, sensor one and sensor (e.g., reference detector 206) and sensor two (e.g., signal detector 208) can measure the intensity of light emitted and reflections generated responsive to emitted light from the first laser source (e.g., at block 402 and block 404) and then sensor one and sensor two measure the intensity of light emitted, and reflections generated responsive to emitted light from the second laser source. It is noted that in embodiments where multiple laser sources are provided, one of the two laser sources may be a low-power source arranged to emit a reference signal while the other laser source may be a high-power source arranged to emit a therapeutic signal. In other embodiments, multiple laser sources each arranged to emit a therapeutic light signal (e.g., light having sufficient energy to ablate or dust a stone, or the like) can be provided.

Continuing to block 406 “determine, at the processor during a time period, a plurality of distances based on the second electrical signal and the first electrical signal, each of the distances corresponding to a distance between the distal end of the optical fiber and at least one of the one or more targets” a plurality of distances between ones of the one or more targets and the distal end of the optical fiber can be determined. For example, computing system 110 can determine the distances (e.g., distances 312, 314, 316, 318, etc.) from the electrical signals received at block 402 and block 404. As another example, processor 904 can execute application instructions 924, which when executed can cause the processor 904 to determine the distances based on the electrical signals received at block 402 and block 404.

Continuing to block 408 “In block 408, method 400determine, at the processor, a pop-dusting efficiency based on the plurality of distances” an efficiency of a pop-dusting procedure can be determined based on the plurality of distances. For example, computing system 110 can determine an efficiency of a pop-dusting procedure based on the distances determined at block 406. As another example, processor 904 can execute application instructions 924, which when executed can cause the processor 904 to determine an efficiency of a pop-dusting procedure based on the distances determined at block 406. As a specific example, the efficiency can be determined based on the number of distances less than a threshold distance determined within a specified period of time. In such an example, the higher the number of distances less than a threshold distance determined at block 406 to occur within the period of time, the higher the efficiency of the pop-dusting procedure.

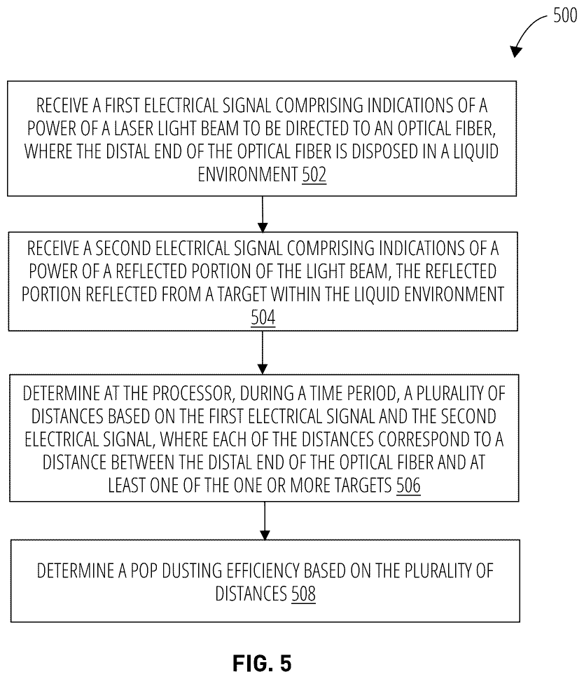

FIG. 5 illustrates a method 500 for determining a pop-dusting efficiency, according to at least one embodiment of the present disclosure. Method 500 can be implemented by a processor of a laser emitting medical device, such as, for example, a processor of lithotripsy procedure system 100. Method 500 will be described with reference to lithotripsy procedure system 100 as well as lasing system 200 and computing environment 900 for clarity of presentation. However, it is noted that method 500 could also be implemented by a laser emitting medical device different from lithotripsy procedure system 100 without departing from the scope of the disclosure.

Method 500 can begin at block 502. At block 502 “receive a first electrical signal comprising indications of a power of a laser light beam to be directed to an optical fiber, where the distal end of the optical fiber is disposed in a liquid environment” an electrical signal can be received, where the electrical signal comprises indications of a power of a laser light to be directed to an optical fiber disposed in a liquid environment. For example, processor 904 can execute application instructions 924 to receive an electrical signal from reference detector 206 comprising an indication of a power of laser light 116.

Continuing to block 504 “receive a second electrical signal comprising indications of a power of a reflected portion of the light beam, the reflected portion reflected from a target within the liquid environment” an electrical signal can be received, where the electrical signal comprises indications of a power of a laser light reflected from a target within the liquid environment. For example, processor 904 can execute application instructions 924 to receive an electrical signal from signal detector 208, where the electrical signal is indicative of a power of reflected laser light 120. As outlined above, reflected laser light 120 is reflected from a target (e.g., stone fragment 304, or the like) within the liquid environment and wherein optical fiber 104 is disposed in environment 122, which is a liquid environment.

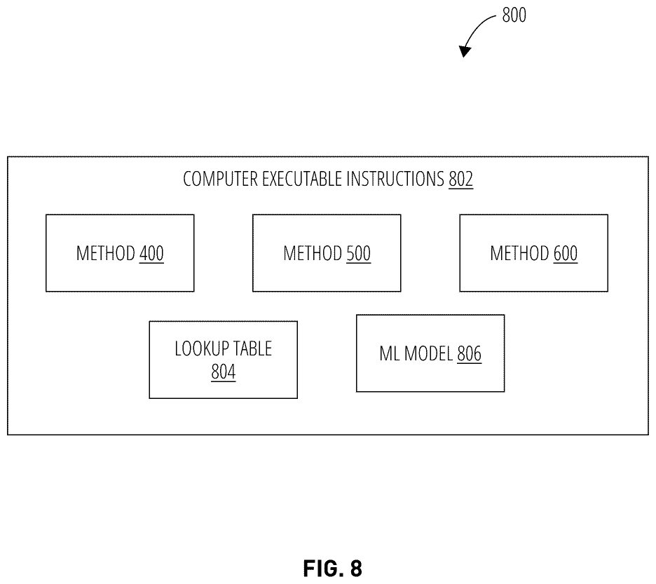

Continuing to block 506 “determine at the processor, during a time period, a plurality of distances based on the first electrical signal and the second electrical signal, where each of the distances correspond to a distance between the distal end of the optical fiber and at least one of the plurality of targets” the distance of a target from the distal end 114 of the optical fiber 104 can be determined based on the electrical signal received at block 502 and the electrical signal received at block 504. For example, memory storage device 906 can comprise a lookup table (e.g., see FIG. 8) correlating a ratio of reflected light to distance of target and processor 904 can execute application instructions 924 to determine from the lookup table the distance of the target based on the determined ratio of emitted and reflected light. In still another example, memory storage device 906 can comprise (e.g., see FIG. 8) a trained machine learning (ML) model (e.g., a trained neural network, or the like) configured to receive as input a ratio of light and infer the distance of the target. In such an example, processor 904 can be configured to execute application instructions 924 to execute the ML model to infer the distance of a target based on the determined ratio of emitted and reflected light.

Continuing to block 508 “determine a pop dusting efficiency based on the plurality of distances” an efficiency of a pop dusting procedure can be determined based on the plurality of distances. For example, memory storage device 906 can comprise a lookup table (e.g., see FIG. 8) correlating a ratio of reflected light to distance of target and processor 904 can execute application instructions 924 to determine from the lookup table the distance of the target based on the determined ratio of emitted and reflected light. In still another example, memory storage device 906 can comprise (e.g., see FIG. 8) a trained machine learning (ML) model (e.g., a trained neural network, or the like) configured to receive as input a ratio of light and infer the distance of the target. In such an example, processor 904 can be configured to execute application instructions 924 For example, it is to be appreciated that as the distance between a target and the distal end of the fiber changes, the ratio of emitted to reflected light may change. As such, the efficiency of the procedure may change. As the targets get closer to the distal end of the optical fiber, the laser incident on the target is more powerful, and more light is reflected into the distal end of the optical fiber. Additionally, as the laser incident on the target is more powerful, the efficiency of the pop-dusting may increase. Accordingly, an efficiency of the pop dusting procedure based on the distances determined at block 506 can be quantified.

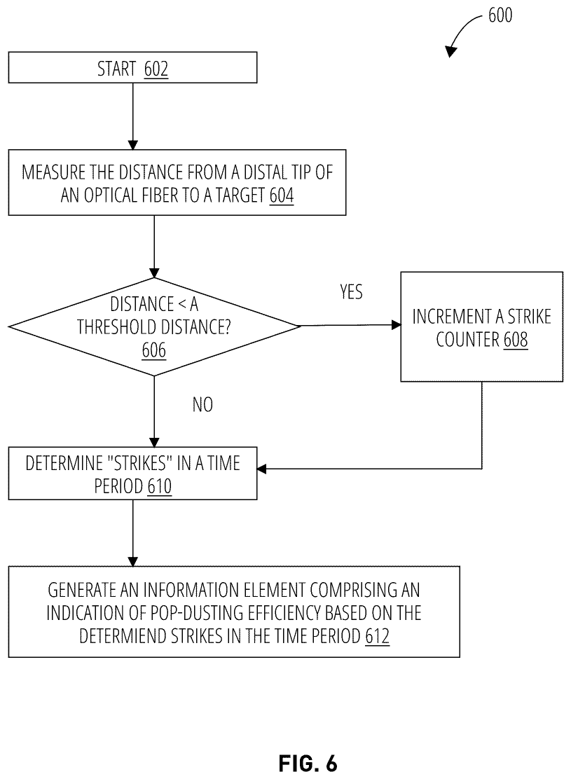

FIG. 6 illustrates a method 600 for determining a pop-dusting efficiency, according to at least one embodiment of the present disclosure. Method 600 can be implemented by a processor of a laser emitting medical device, such as, for example, a processor of lithotripsy procedure system 100. Method 600 will be described with reference to lithotripsy procedure system 100 as well as lasing system 200 and computing environment 900 for clarity of presentation. However, it is noted that method 600 could also be implemented by a laser emitting medical device different from lithotripsy procedure system 100 without departing from the scope of the disclosure.

Method 600 can begin at block 602. At block 602 “start” the pop-dusting efficiency determination process of method 600 can begin. Continuing to block 604 “measure the distance from a distal tip of an optical fiber to a target” where the distance from the distal tip of an optical fiber to a target can be measured. For example, processor 904 can execute application instructions 924 to cause lasing system 200 to measure the distance (e.g., distance 312, 314, 316, etc.) between the distal end 114 of optical fiber 104 and a target (e.g., stone fragment 304, 306, 308, etc.).

Continuing to decision block 606 “distance < a threshold distance?” a determination of whether the distance is less than a threshold distance can be made. For example, processor 904 can execute application instructions 924 to cause lasing system 200 to determine whether the distance measured at block 604 is less than a threshold distance. In some embodiments, the threshold distance can be 2 millimeters. From decision block 606, method 600 can continue to either block 608 or block 610. Method 600 can continue from decision block 606 to block 608 based on a determination at decision block 606 that the distance is less than the threshold while method 600 can continue from decision block 606 to block 610 based on a determination at decision block 606 that the distance is not less than a threshold.

At block 608 “increment a strike counter” a strike counter can be incremented. For example, processor 904 can execute application instructions 924 to increment a memory location, data structure, or register value comprising a strike counter or a count of strikes over time. At block 610 “determine strikes in a time period” several strikes in a time period can be determined based on the strike counter. For example, processor 904 can execute application instructions 924 to determine the number of strikes occurring over a time period (e.g., 0.3 seconds, 0.5 seconds, 0.7 seconds, or the like). In some embodiments, the strikes over (or in) a time period can be determined for the trailing time period (e.g., strikes in the last 1 second, or the like).

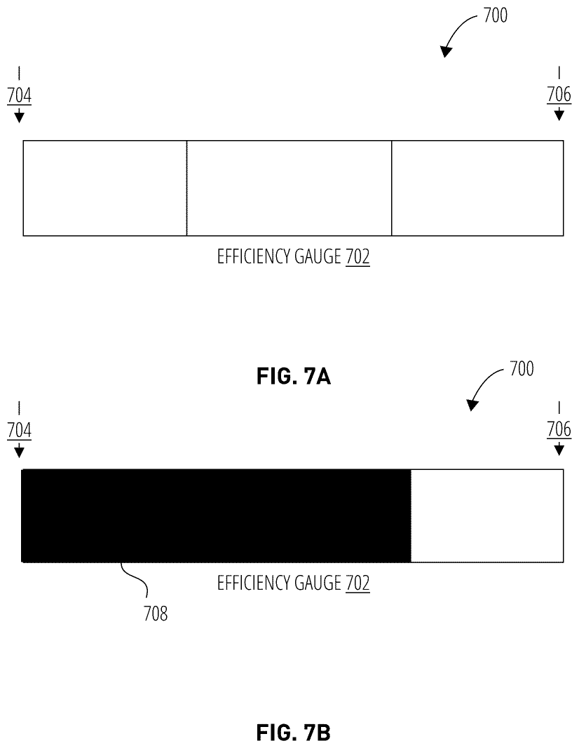

Continuing to block 612 “generate an information element comprising an indication of pop-dusting efficiency based on the determined strikes in the time period. For example, processor 904 can execute application instructions 924 to generate a graphical information element (e.g., graphical user interface (GUI) element, or the like) indicative of the efficiently of a pop-dusting procedure based on the determined strike count over a time period. Example graphical information elements are given in FIG. 7A and FIG. 7B. Further, processor 904 can execute application instructions 924 to cause the generated graphical information element to be displayed on a display (e.g., of laser console 102, or the like).

Accordingly, method 600 provides to compare determined distances to a predetermined distance threshold to derive a “strikes per second” score. The strikes per second score can be used to determine the efficiency of the pop-dusting procedure. For example, referring to FIG. 3A to 3D, where the stone fragments 304 (etc.) are large the number of strikes per second will be higher, which could imply that the pop-dusting procedure is proceeding efficiently. As the stone fragments are broken down and dusted (e.g., FIG. 3D, FIG. 3E, or the like) the number of strikes over a set time period will decrease. Specifically, as the stone fragments (e.g., stone fragment 304a, etc.) become small enough, too small a portion of the laser light 116 will be reflected to measure a distance and/or the distance will be measured as larger than the threshold and the number of strikes over the given time period will decrease. This can indicate that either the pop-dusting procedure is complete (e.g., strikes per second below a lower threshold of the efficiency is decreasing strikes per second between the lower threshold and a higher threshold.

FIG. 7A demonstrates a preferred embodiment of a graphical information element 700 in which an efficiency gauge 702 that is configured to dynamically display the pop-dusting efficiency in real-time. The efficiency gauge 702 may include a colored bar (or other graphical indication such as patterned, or the like) that is adjusted dynamically based on the determined pop-dusting efficiency to indicate to a user the average efficiency of the treatment. In some embodiments, one end of the efficiency gauge can indicate greater efficiency 706 while the opposite end can indicate lower efficiency 704.

FIG. 7B illustrates 700 where the efficiency gauge 702 is adjusted (e.g., dynamically during a procedure) to indicate the currently determined pop-dusting efficiency. For example, efficiency gauge 702 is depicted partially filled in (e.g., with a color, with a pattern, or the like) to indicate to the user the current efficiency of the treatment. It is to be appreciated, that where efficiency gauge 702 is completely colored, shaded, or patterned, graphical information element 700 indicates a high efficiency while where efficiency gauge 702 is not colored, shaded, or patterned, graphical information element 700 indicates a low efficiency. Further, where the efficiency gauge 702 is partially colored, shaded, or patterned, some efficiency between high and low is indicated.

FIG. 8 illustrates computer-readable storage medium 800. Computer-readable storage medium 800 may comprise any non-transitory computer-readable storage medium or machine-readable storage medium, such as an optical, magnetic or semiconductor storage medium. In various embodiments, computer-readable storage medium 800 may comprise an article of manufacture. In some embodiments, computer-readable storage medium 800 may store computer executable instructions 802 with which circuitry (e.g., computing system 110, processor 904, or the like) can execute. For example, computer executable instructions 802 can include instructions to implement operations described with respect to method 400, method 500, or method 600. Further, computer executable instructions 802 can store data structures or other information, such as, for example, lookup table 804, or ML Model 806, or graphical information element 700. Examples of computer-readable storage medium 800 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 802 may include any suitable type of code, such as source code, compiled code, interpreted code, executable code, static code, dynamic code, object-oriented code, visual code, and the like.

FIG. 9 is a block diagram of a computing environment 900 including a computer system 902 for implementing embodiments consistent with the present disclosure. In some embodiments, the computing environment 900, or portion thereof (e.g., the computer system 902) may comprise or be comprised in a laser system (e.g., the computing system 110 of the lithotripsy procedure system 100 can embody portions of the computing environment 900). Accordingly, in various embodiments, computer system 902 may determine an efficiency of a pop-dusting procedure as described above.

The computer system 902 may include a central processing unit (“CPU” or “processor”) 904. The processor 904 may include at least one data processor for executing instructions and/or program components for executing user or system-generated processes. A user may include a person, a person using a device such as those included in this disclosure, or another device. The processor 904 may include specialized processing units such as integrated system (bus) controllers, memory management control units, floating point units, graphics processing units, neural processing units, digital signal processing units, etc. The processor 904 may be disposed in communication with input devices 914 and output devices 916 via I/O interface 912. The I/O interface 912 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE), WiMAX, or the like), etc.

Using the I/O interface 912, computer system 902 may communicate with input devices 914 and output devices 916. In some embodiments, the processor 904 may be disposed in communication with a communications network 920 via a network interface 910. In various embodiments, the communications network 920 may be utilized to communicate with a remote memory storage device 906, such as for accessing look-up tables, performing updates, or utilizing external resources. The network interface 910 may communicate with the communications network 920. The network interface 910 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc.

The communications network 920 can be implemented as one of the different types of networks, such as intranet or Local Area Network (LAN), Closed Area Network (CAN) and such. The communications network 826 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 920 may include a variety of network devices, including routers, bridges, servers, computing devices, storage devices, etcetera. In some embodiments, the processor 904 may be disposed in communication with a memory storage device 906 via a storage interface 908. The storage interface 908 may connect to memory storage device 906 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etcetera.

Furthermore, memory storage device 906 may include one or more computer-readable storage media utilized in implementing embodiments consistent with the present disclosure. Generally, a computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media.

The memory storage device 906 may store a collection of program or database components, including, without limitation, an operating system 922, an application instruction 924, and a user interface elements 926. In various embodiments, the operating system 922 may facilitate resource management and operation of the computer system 902. Examples of operating systems include, without limitation, APPLE® MACINTOSH® OS X®, UNIX®, UNIX-like system distributions (E.G., BERKELEY SOFTWARE DISTRIBUTION® (BSD), FREEBSD®, NETBSD®, OPENBSD, etc.), LINUX® DISTRIBUTIONS (E.G., RED HAT®, UBUNTU®, KUBUNTU®, etc.), IBM®OS/2©, MICROSOFT® WINDOWS® (XP®, VISTA©/7/8, 10 etc.), APPLE® IOS®, GOOGLE™ ANDROID™, BLACKBERRY® OS, or the like.

The application instructions 924 may include instructions that when executed by the processor 904 cause the processor 904 to perform one or more techniques, steps, procedures, and/or methods described herein, such as to determine a distance of a target disposed in a liquid environment (e.g., environment 122) based on a plurality of light signals (e.g., signal detector 208 and/or reference detector 206) formed by the laser light.

The user interface elements 926 may facilitate display, execution, interaction, manipulation, or operation of program components through textual or graphical facilities. For example, user interfaces may provide computer interaction interface elements on a display system operatively connected to the computer system 902, such as cursors, icons, checkboxes, menus, scrollers, windows, widgets, etcetera. The user interface elements 926 may be employed by application instructions 924 and/or operating system 922 to provide, for example, a user interface with which a user can interact with computer system 902. In some embodiments, the user interface elements 926 may be displayed on a display.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A system for a laser console, comprising:
a processor; and
a memory device coupled to the processor, the memory device having instructions stored thereon, which instructions when executed by the processor, cause the system to:
receive a first electrical signal generated by a first light sensor, the first electrical signal comprising an indication of a power of a light received at the first light sensor, wherein the light received at the first light sensor corresponds to laser light generated by a laser source and emitted from a distal end of an optical fiber towards one or more targets;
receive a second electrical signal generated by a second light sensor, the second electrical signal comprising an indication of a power of a light received at the second light sensor, wherein the light received at the second light sensor corresponds to a laser light reflected from at least one or more targets;
determine, during a time period, a plurality of distances based on the second electrical signal and the first electrical signal, each of the distances corresponding to a distance between the distal end of the optical fiber and at least one of the one or more targets; and
determine a pop-dusting efficiency based on the plurality of distances.

2. The system of claim 1, wherein the optical fiber is coupled to the laser console.

3. The system of claim 1, the instructions, when executed by the processor, further cause the system to:
identify a reference in a lookup table corresponding to the distance, wherein the lookup table is stored in a memory coupled to the processor, and wherein the lookup table correlates a distance of the target with the first electrical signals and second electrical signals; and
determine the distance of the target from the distal end of the optical fiber based on the reference.

4. The system of claim 1, wherein the laser console comprises a lasing system comprising:
the light sensor;
a laser source arranged to generate the laser light; and
a beam splitter arranged to direct a portion of the laser light from the laser source to the optical fiber and arranged to direct the laser light reflected from the one or more targets to the light sensor.

5. The system of claim 4, wherein the laser console comprises the second light sensor and wherein the beam splitter is further arranged to direct a portion of the laser light from the laser source to the second light sensor.

6. The system of claim 4, wherein the laser source comprising either a Holmium-based lasing medium or a Thulium-based lasing medium.

7. The system of claim 4, wherein the laser console further comprises an optical head comprising at least a lens arranged to couple the laser light to the optical fiber.

8. The system of claim 4, wherein the lasing system further comprises a second laser source and wherein the instructions, when executed by the processor, further cause the system to:
receive a third electrical signal generated by the first light sensor, the third electrical signal comprising an indication of a power of a light received at the first light sensor that corresponds to laser light generated by the second laser source and emitted from the distal end of the optical fiber towards the one or more targets;
receive a fourth electrical signal generated by the second light sensor, the fourth electrical signal comprising an indication of a power of a light received at the second light sensor that corresponds to laser light reflected from at least one of the one or more targets; and
determine, at the processor during the time period, the plurality of distances based on the first electrical signal, the second electrical signal, the third electrical signal and the fourth electrical signal.

9. The system of claim 1, the instructions, when executed by the processor, further cause the system to execute a machine learning (ML) model to generate an inference of the distance of a target from the distal end of the optical fiber, wherein the machine learning model is executed with at least the first electrical signal and second electrical signals as input.

10. The system of claim 1, wherein the optical fiber is arranged to be inserted through a working channel of a ureteroscope.

11. A computer-readable memory storage device comprising a plurality of instructions, which when executed by a processor of a medical laser console, cause the medical laser console to:
receive a first electrical signal generated by a first light sensor, the first electrical signal comprising an indication of a power of a light received at the first light sensor, wherein the light received at the first light sensor corresponds to laser light generated by a laser source and emitted from a distal end of an optical fiber towards one or more targets;
receive a second electrical signal generated by a second light sensor, the second electrical signal comprising an indication of a power of a light received at the second light sensor, wherein the light received at the second light sensor corresponds to a laser light reflected from at least one or more targets;
determine, during a time period, a plurality of distances based on the second electrical signal and the first electrical signal, each of the distances corresponding to a distance between the distal end of the optical fiber and at least one of the one or more targets; and
determine a pop-dusting efficiency based on the plurality of distances.

12. The computer-readable memory storage device of claim 11, the instructions, when executed by the processor further cause the medical laser console to:
determine the distance of the target from the distal end of the optical fiber;
identify in a lookup table a reference corresponding to a predetermined distance threshold;
compare the distance of the target to the predetermined distance threshold;
count distances less than the threshold as strikes; and
update the pop-dusting efficiency based on strikes per second.

13. The computer-readable memory storage device of claim 11, wherein as the plurality of distances decreases, the pop-dusting efficiency increases.

14. The computer-readable memory storage device of claim 11, wherein as the plurality of distances increases, the pop-dusting efficiency decreases.

15. The computer-readable memory storage device of claim 11, the medical laser console comprising a display and wherein the instructions, when executed by the processor further cause the medical laser console to:
generate one or more graphical information elements comprising indications of the pop-dusting efficiency; and
cause the display to display the graphical information elements as part of a user interface.

16. The computer-readable memory storage device of claim 15, wherein the one or more graphical information elements comprise a pop-dusting efficiency gauge, which is configured to display the pop-dusting efficiency.

17. A computer implemented method, comprising:
receiving, at a processor, a first electrical signal generated by a first light sensor, the first electrical signal comprising an indication of a power of a light received at the first light sensor, wherein the light received at the first light sensor corresponds to laser light generated by a laser source and emitted from a distal end of an optical fiber towards one or more targets;
receiving, at the processor, a second electrical signal generated by a second light sensor, the second electrical signal comprising an indication of a power of a light received at the second light sensor, wherein the light received at the second light sensor corresponds to a laser light reflected from at least one or more targets;
determining, at the processor during a time period, a plurality of distances based on the second electrical signal and the first electrical signal, each of the distances corresponding to a distance between the distal end of the optical fiber and at least one of the one or more targets; and
determining, at the processor, a pop-dusting efficiency based on the plurality of distances.

18. The compute implemented method of claim 17, wherein the lasing system further comprises a second laser source, the method comprising:
receiving, at a processor, a third electrical signal generated by the first light sensor, the third electrical signal comprising an indication of a power of a light received at the first light sensor that corresponds to laser light generated by the second laser source and emitted from the distal end of the optical fiber towards the one or more targets;
receiving, at the processor, a fourth electrical signal generated by the second light sensor, the fourth electrical signal comprising an indication of a power of a light received at the second light sensor that corresponds to laser light reflected from at least one of the one or more targets; and
determining, at the processor during the time period, the plurality of distances based on the first electrical signal, the second electrical signal, the third electrical signal and the fourth electrical signal.

19. The computer implemented method of claim 17, comprising:
identifying, by the processor, a reference in a lookup table corresponding to the distance, wherein the lookup table is stored in a memory coupled to the processor, and wherein the lookup table correlates a distance of the target with the first electrical signals and second electrical signals; and
determining the distance of the target from the distal end of the optical fiber based on the reference.

20. The computer implemented method of claim 17, comprising:
determining the distance of the target from the distal end of the optical fiber;
identifying in a lookup table a reference corresponding to a predetermined distance threshold;
comparing the distance of the target to the predetermined distance threshold;
counting distances less than the threshold as strikes; and
updating the pop-dusting efficiency based on strikes per second.
